(19) United States
(12) Patent Application Publication
(10) Pub. No.: US 2019/0304596 A1
(43) Pub. Date: Oct. 3, 2019

(54) Use Of Historic And Contemporary Tracking Data To Improve Healthcare Facility Operations

(71) Applicant: Tagnos, Inc., Irvine, CA (US)

(72) Inventor: Jagadesh Appla Padala, Irving, TX (US)

(21) Appl. No.: 16/190,681

(22) Filed: Nov. 14, 2018

Related U.S. Application Data
(60) Provisional application No. 62/585,868, filed on Nov. 14, 2017.

(51) Int. Cl. G16H 40/20; G06Q 10/06; G06N 20/00

(52) U.S. Cl. CPC G16H 40/20; G06N 20/00; G06Q 10/06375

(57) ABSTRACT

Systems, methods, kits, and devices are contemplated for improving healthcare and healthcare activities within a healthcare facility. Historical and live tracking data is used to train models for predicting activity outcomes in the facility, identifying outcome relevant variables to develop pilot programs designed to favorably change the predicted outcome, and implementing the pilot programs to yield an actual outcome, preferably within a tolerable margin from a desired outcome. Systems and methods for developing a predictive model, or easing healthcare administrators in developing accurate predictive models or improving predictive models, are also contemplated.

This application claims priority to copending U.S. provisional application No. 62/585,868, filed Nov. 14, 2017, which is incorporated by reference herein.

### FIELD OF THE INVENTION

The field of the invention is orchestrating healthcare facilities and activities.

### BACKGROUND

Managing, orchestrating, or optimizing work flows that involve multiple prerequisites, tasks, participants, or resources with disparate lead and lag times is a substantial problem for large facilities or enterprises, and as such an area of great interest and development. However, designing, improving, or implementing such workflows is incredibly complicated and often yields suboptimal or unacceptable results due to cost-prohibitive or insurmountable challenges. Such complications are compounded in industries where multiple, concurrent operations are performed that require overlapping resources with shared upstream or downstream tasks, all occurring within a confined space (e.g., manufacturing facilities, construction sites, healthcare facilities, etc). The burden of designing, improving, or implementing workflows grows exponentially in healthcare facilities, where modifying workflows can have life or death affects on patients and where the results of individual activities (e.g., surgery, treatment outcome, etc) are largely unknown or unpredictable, and can be interrupted by unanticipated emergencies.

Efforts have been made to address the dilemmas faced in managing, orchestrating, or optimizing complex workflows in healthcare facilities. For example, U.S. Pat. No. 7,492,266 to Bhavani et al (the '266 patent), and likewise US Publication No. 2009/0051546 to Bhavani (the '546 application), teaches improving workflows using VoIP and RFID tags to improve communication between doctors, nurses, and healthcare resources and providing updated information regarding resources. However, while simply improving communication and updating information may improve performance of workflows, they offer little or no aid in the design of workflows, or the improvement and optimization of those workflows. Further, the teachings of the '266 patent require doctors or nurses to actively initiate the system and communicate, therefore lacking the benefits of passive, background, or automated management systems.

These and all other publications referenced herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

US Publication No. 2009/0315735 to Bhavani (the '745 application) improves managing patients at hospitals by assigning a series of progressive stages to a patient for their treatment (i.e., registration, waiting, examination, etc) and tracking patients and resources used in the stages as the patient progresses through the stages, and running reports to update the patient's status. But teachings of the '745 application fail to appreciate that computer models trained on historical health care facility data can be advantageously combined with live tracking data, as well as upstream and downstream workflow requirements, to predict likely outcomes of a current workflow, identify workflow variables that effect the predicted outcome, and implement modifications of those variables, causing a new outcome.

Thus, there remains a need for methods and systems that utilize historic and contemporary tracking data to build outcome predictive models for activities in a health care activity that are used implement changes in a workflow to change a predicted outcome of the workflow.

### SUMMARY OF THE INVENTION

The inventive subject matter contemplates kits, devices, systems, and methods for improving healthcare and healthcare activities within a health care facility using historical and live tracking data to train models for predicting activity outcomes, developing pilot programs designed to favorably change the predictive outcome, and implementing the pilot programs to yield an actual outcome, preferably within a tolerable margin from a desired outcome.

Portable electronic transmitters (e.g., RFID, RTLS, IoT, etc) are used to track locations and movements of at least some healthcare assets in the healthcare facility, thereby accumulating historic tracking data and producing contemporary (e.g., live, etc) tracking data. The historic tracking data and contemporary tracking data are used in combination with a healthcare outcome of a previous healthcare event (e.g., workflow, treatment, etc) and at least one facility-operating model to predict a future healthcare outcome of a future healthcare event. A pilot program is then conducted by altering a planned future activity within the healthcare facility (e.g., a variable of the activity), thereby producing an actual outcome that is different from the predicted outcome. The facility-operating model is then modified to incorporate at least one alteration from the pilot program, and the modified facility-operating model is implemented within the healthcare facility.

At least some of the portable electronic transmitters are active transmitters, but other portable electronic transmitters can be passive transmitters, or both. Most of the contemporary tracking data that is produced (and preferably at least some of the historical tracking data) is granular to a level of an individual healthcare asset within a type or category of healthcare assets (e.g., gurney A, MRI scanner B, surgeon C, cardiologist D, nurse practitioner E, operating room F, etc). The contemporary tracking data generally tracks at least one of (a) an individual healthcare professional among a type of healthcare professional (e.g., doctor A, cardiologist, B, general practitioner C, nurse D, etc) or (b) one or more pieces of healthcare equipment within one or more types of healthcare equipment (e.g., scalpel A, bed B, etc).

In some embodiments at least one of the facility-operating models is at least partially developed by training one or more machine learning algorithms on at least part (preferably all) of the historical tracking data. The facility-operating model utilizes machine learning (e.g., at least one algorithm trained on at least one historical data set) to automatically compare at least one of (a) the actual outcome with the predicted outcome or (b) the pilot program with the actual outcome. It should be appreciated that further using at least a second facility-operating model (e.g., employs a different methodology from the first facility-operating model, is trained on a different data set, includes different limitations or degrees of freedom, a modified version of the first-operating model, etc) to comparison check a predictive accuracy of the first facility-operating model is beneficial. Such comparison can be used to adjust the first facility-operating model (e.g., adding a variable, removing a variable, adjusting the weight of a variable, reclassifying a variable, etc), or another facility-operating model, or to create a new model (e.g., combining features of multiple models, removing features of a model, adding new features, etc). Viewed from another perspective, it should be appreciated that the future activity (e.g., a patient variable, an activity variable, a time variable, a healthcare resource variable, etc) is altered based on a feature (e.g., a predicted outcome, a predicted risk factor, a variable identified as relevant, a variable identified as irrelevant, etc) of at least one facility-operating model.

Thus it should be appreciated that in some embodiments, the future activity is altered directly based on a second facility-operating model (e.g., a feature of the second model), or indirectly based on the second facility-operating model (e.g., a feature of the second model is combined with another model). It should also be appreciated that the future activity can be altered by part of a first model and part of a second model, either concurrently or at separated times.

It is contemplated that at least one of (a) a status of one of the healthcare assets, (b) a status of a condition precedent to the future activity (e.g., condition is required, planned, incidental, unexpected, etc), or (c) a status of other activities in the healthcare facility is used to predict the future healthcare outcome (e.g., variable added to a facility-operating model, weight of a variable changed, training model with status, combining status with contemporary tracking data used to predict outcome, etc). In some embodiments altering the future activity entails at least one of (a) replacing a one of the healthcare assets with a second healthcare asset, (b) requisitioning an additional healthcare asset, or (c) withdrawing one of the healthcare assets from utilization in the future activity. Altering the future activity can include a number of additional or alternative changes, for example rescheduling the future activity, canceling the future activity, or prioritizing the future activity over other future activities. Viewed from another perspective, the pilot program can additionally or alternatively include rescheduling at least one other activity planned in the healthcare facility or reallocating healthcare assets related to one or more other activities.

The inventive subject matter further contemplates devices, systems, kits, and methods for orchestrating physical activities taking place in a healthcare facility. One or more historical data sets including a plurality of historic variables and a physical activity result associated with at least some of the variables are accessed. An experimentation model is trained (e.g., via machine learning, neural network, artificial intelligence, etc) on the historical data set to relate the plurality of historic variables to the physical activity result. One or more contemporary data sets (e.g., live, real-time, near real-time, etc) related to a first plurality of contemporary variables associated with a scheduled physical activity are collected. A predicted result for the scheduled physical activity is generated by applying the contemporary data set to the experimentation model.

The predicted result is compared to a threshold (e.g., user set, default, generated by model, etc). Should the predicted result exceed the threshold, a set of modified variables with a modification to one or more of the plurality of contemporary variables is created such that, when the set of modified variables is applied to the experimentation model, a new predicted result is generated that satisfies the threshold. The set of modified variables are then physically implemented to physically perform the scheduled activity in the facility. A new data set is recorded including (a) the implemented set of modified variables and (b) an actual result of the scheduled physical activity. The new data set, preferably in combination with the historical data set, is then used to train a new experimentation model, preferably with improved accuracy of predicting results of activities. In preferred embodiments, the new experimentation model is implemented in the healthcare facility to orchestrate at least one physical activity.

In some embodiments the plurality of historical variables relates to at least two of (a) a healthcare asset data, (b) a downstream condition, (c) a patient status, (d) an upstream result (e.g., report result, scan result, referral, recommendation, patient health event, health insurance restriction, delayed healthcare asset, delayed patient, facility delay, etc), or (e) another activity that occurred in the healthcare facility. It should be appreciated that the healthcare asset data includes at least one of a location, a condition (e.g., clean, dirty, used, broken, repaired, operational, ready, charged, etc), an identity (e.g., type of asset, individual asset within type of asset, name of asset, etc), a cost, a time element (e.g., lag time, lead time, requires delay, ready now, available later, etc), or a utilization rate of a healthcare asset. Likewise, the downstream condition is preferably at least one of a lab report, a blood test, a radiology report, a confirmation, a denial, or availability of healthcare assets. Further, the patient status is preferably at least one of late, early, prepared, a location, or a health condition status of a patient, but further patient status are contemplated.

It is preferred that the physical activity result in a patient outcome, an activity duration, an activity delay, an impact on concurrent or prospective activities in the healthcare facility, an activity cost, an activity related litigation, or a combination thereof. It should be appreciated that the experimentation model can advantageously determine a risk factor associated with each (or at least some) type of historical variables, but such risk factors can also be user defined. In some embodiments, the set of modified variables does not include modification of a variable type associated with a risk factor that exceeds a risk threshold (e.g., user defined, default, model generated, etc). For example, where the model determines modification of a variable type has a sufficient likelihood (e.g., 30%, 60%, 90%) to yield a negative patient outcome result (e.g., injury, death, reduced health status, etc), then that variable type will not be modified.

Likewise, it is contemplated the experimentation model can identify a set of key variables that have a significant impact on the predicted result (e.g., variable is determinative, impacts 100% of outcomes, impacts 80% of outcomes, impacts 50% of outcomes, impacts 20% of outcomes, changes outcome by 80%, changes outcome by 60%, changes outcome by 50%, changes outcome by 40%, changes outcome by 30%, changes outcome by 20%, changes outcome by 10%, changes outcome by 5%, etc). It is preferred the set of modified variables includes modification to at least one of the key variables identified, such modification producing a predicted result within a tolerance from the desired outcome. Similarly, it is contemplated the experimentation model can identify a set of insignificant variables that have minimal impact on the predicted result (e.g., de minimis, impacts less than 5% of outcomes, impacts less than 2% of outcomes, impacts less than 1% of outcomes, changes outcome by less than 5%, changes outcome by less than 2%, changes outcome by less than 1%, changes outcome by less than 0.5%, etc). In such embodiments, the insignificant variables are preferably reduced (e.g., assigned coefficient >1) or eliminated (e.g., assigned coefficient of 0) when generating the new predicted result, training the new experimentation model, implementing the new experimentation model in the healthcare facility, or a combination thereof.

In some embodiments, the contemporary tracking data, the historic tracking data, or both, include movement data of a patient, for example indicating whether the patient is pacing or displaying other agitated behavior (e.g., repetitive motion, frequent inquiries to staff, aggressive behavior, etc), is in an assigned location, or is bothering other patients or healthcare staff in the facility. This movement data of the patient is used to indicate the patient is anomalous (e.g., agitated, unstable mental state, potentially dangerous, requires additional attention, etc), to predict the patient is approaching an anomalous state (e.g., the patient is becoming angry, is nervous, is sleep deprived, etc), or predict a future event at the facility will push the patient toward an anomalous state (e.g., fear of needles, white-coat fever, other susceptibility, etc). Preferably, a future activity or activities within the healthcare facility is altered or added to mitigate the anomalous patient, for example administering medication without a needle, administering a sedative to the patient, providing mental health relief to the patient, alerting security, etc. Other patient data is preferably considered in addition movement data, for example the age of the patient, to discount what appears to be anomalous movements as indicating an anomalous patient, for example that a child tends to move around quite a bit but is not considered to be anomalous or present a risk to the facility.

It is also contemplated that the historic tracking data, the contemporary tracking data, or both include a data set regarding a specific healthcare professional, for example a specific doctor (e.g., neurologist A, surgeon B, proctologist C, etc) or a specific nurse (e.g., RN 1, LPN 2, CNS 3, NP 4, etc). The data set regarding the specific healthcare professional is used to compile a profile of that specific healthcare professional. Such data set would include data indicating, for example, the professional's time efficiency, standard of care, bedside manner, cost efficiency, success rate, risk factor, and impact on patient health. The data set preferably logs at least one of the number of visits to a patient, the frequency of visits with a patient, the number of minutes spent with a patient, the delays in visiting a patient, the amount of time spent away from a patient, duration of procedure, the relative improvement in patient health, complaints made by the patient about the specific professional, or complaints made by other facility staff about the specific professional. It is contemplated that such data at least partially affects the outcome predicted by the inventive subject matter, and preferably a future activity of the specific healthcare professional is modified to produce an actual outcome different from the predicted outcome, for example reassignment of the professional, additional training for the professional, termination of the professional, extended time off or paid time off for the professional, etc.

Systems and methods are contemplated for implementing a pilot program, for example at a healthcare facility. A desired outcome (or goal, objective, etc) for the pilot program is assigned, for example a time for a procedure, an efficiency rate of healthcare professionals, an efficiency rate of healthcare assets, a minimum patient wait time, a minimum patient treatment time, patient treatment efficiency, a case length (e.g., surgery, etc) or other outcomes or goals that a favorable in a particular enterprise, for example a healthcare facility. As should be appreciated, the outcome is generally associated with a number of variables, sometimes hundreds, thousands, tens of thousands, hundreds of thousands, or even millions of variables, at least some of which are used in the inventive subject matter.

Using at least part of a historic dataset (e.g., years of prior data, months of prior data, weeks of prior data, days of prior data, hours of prior data, minutes of prior data, etc) or a contemporary dataset (e.g., minutes old, seconds old, near real-time, real-time, known projected, worst case projected, best case projected, speculative, worst case speculative, best case speculative, etc), weights are assigned to the variables (e.g., at least some of the variables associated with the outcome, half of the variables, most of the variables, all of the variables, etc) with respect to impact on the outcome. For example, if a variable significantly impacts the outcome, or the accuracy of a model predicting the outcome, the variable can be weighted higher than variables that have little or no impact on the outcome (or predictive accuracy, or both). Accordingly, high impact variables are weighted toward or at the upper threshold of a scale (e.g., 1 or near 1 on a scale of 0 to 1, etc), while little or no impact variables are rated toward or at the lower threshold of the scale (e.g., 0 or near 0 on a scale or 0 to 1, etc). In some embodiments, the variables are weighted (or narrowed) via a least absolute shrinkage and selection operator (LASSO) analysis, or other appropriate statistical method, alone or in combination.

In the alternative, or in addition thereto, a set of features is defined among at least some variables in the historic or contemporary datasets, or both. The features are defined such that each feature has an impact relative to the outcome. The features can be defined with user supervision, with unsupervised machine learning, or a combination thereof, but can also be predefined. Preferably, at least some of the features are used to construct a tree, for example a learning decision tree, classification and regression tree, etc.

A predictive model (preferably a plurality thereof) for the pilot program is constructed using the weighted plurality of variables and the set of features. While the model(s) can have preset limitations, it is contemplated that a user can define various limitations of the model. For example, a user can set the maximum (or minimum, or both) number of features to be considered, the maximum (or minimum, or both) depth of a tree, the minimum (or maximum, or both) sample size to be considered, the scaled range of weights assigned (e.g., LASSO alpha 0.1 to 1, LASSO alpha 0, etc), and parameters of neural networks to be used.

While it is contemplated that a single predictive model is constructed, a user can define the maximum number of models to be constructed (e.g., no more than 10, 12, 20, 30, 40, 50, 100, 1000, 10000, more than 100000), the maximum runtime for each model or time to construct and cross validate a plurality of models (e.g., no more than 0.1, 1, 5, 10, 12, 20, 30, 40, 60, more than 90 minutes), as well as the number of folds used to cross-validate each model (e.g., 2, 3, 4, 5, 6, 10, 15, 20, or more than 30 folds). Viewed from another perspective, systems and methods of the inventive subject matter construct a plurality of predictive models using contemporary and historical data with weighted variables and defined features, and cross validate those models to select a validated model (e.g., model with highest predictive accuracy, fastest runtime model, model with smallest margin of error, etc). While it is contemplated that the validated model has the greatest accuracy and is used to then design and implement the pilot program, in some embodiments the validated model is further modified before designing the pilot program, for example by modifying a weight or a feature to alter (preferably improve) the accuracy of the validated model in predicting the outcome, yielding an improved validated model.

The validated model (or improved validated model) is used to design a pilot program to implement at the enterprise (e.g., healthcare facility), for example selecting values for the variables considered by the validated model that yield an output closest to the desired outcome, modifying the contemporary value for at least one variable to bring the output of the validated model closer to the desired outcome, etc. For example, the validated model can be used to set a range of values for at least some of the plurality of variables (e.g., healthcare professionals have no more than one consecutive shift over three days, having between two and ten on-call staff at all times, no more than four simultaneous procedures in a single department, do not assign healthcare asset A and B to the same procedure, do not assign healthcare asset A without healthcare asset C, etc) that increases probability of the outcome or brings an output of the model closer to the outcome.

The inventive subject matter can be applied to continuously (or periodical, on-demand, etc) predict census for various departments of an enterprise. For example, the ratio of staff hours per patient can be calculated for an emergency room of a health facility, allowing ER managers to adjust staffing to reach a desired ratio, for example 3.05 staff hours per patient. Past data can be used to train a model to predict future requirements. For example, past data showing over a 7 day period, the ER averaged 200 patients at a time with 10 nurses staffing, and yielding a staff hour to patient ratio of 3.5, and data for the previous 24 hours showing an average of 210 patients with 10 nurses and a ratio of 3.8 staff hours per patient can be used to predict patient census, staffing, and staff hour to patient ratios for future time periods, for example consecutive 12 hour shafts from 6 am to 6 pm and 6 pm to 6 am. For example, such data can be used to predict a 6 am to 6 pm shift will service 230 patients on a given day, with 10 assigned nurses, yielding a 3.9 staff hour to patient ratio. Thus, the system enables an ER manager to increase the nurse staffing during the 6 am to 6 pm shift. Likewise, the model predicts the 6 pm to 6 am shift will service 200 patients with 10 nurses on staff, yielding a staff hour to patient ratio of 3. This informs an ER manager that staffing during the 6 pm to 6 am shift is appropriate to meet the goal staff hour to patient ratio of 3.05.

Descriptions throughout this document include information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary. The recitation of ranges of values herein is merely intended to serve as a shorthand method of referring individually to each separate value falling within the range. Unless otherwise indicated herein, each individual value is incorporated into the specification as if it were individually recited herein.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various objects, features, aspects and advantages of the inventive subject matter will become more apparent from the following detailed description of preferred embodiments, along with the accompanying drawing figures in which like numerals represent like components.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

### BRIEF DESCRIPTION OF THE DRAWING

FIGS. 1A and 1B depict sample data tables considered in the inventive subject matter.

FIG. 2 depicts a user display of the inventive subject matter.

FIG. 3 depicts a sample workflow of the inventive subject matter.

FIG. 4 depicts another sample workflow of the inventive subject matter.

FIG. 5 depicts another user display of the inventive subject matter.

FIG. 6 depicts yet another user display of the inventive subject matter.

FIG. 7 depicts still another user display of the inventive subject matter.

FIG. 8 depicts yet another user display of the inventive subject matter.

FIG. 9 depicts still another user display of the inventive subject matter.

FIG. 10 depicts yet another user display of the inventive subject matter.

FIG. 11 depicts a graph ranking variables considered by the inventive subject matter.

FIG. 12 depicts logloss of models built and validated by the inventive subject matter.

### DETAILED DESCRIPTION

The inventive subject matter contemplates kits, devices, systems, and methods for improving healthcare and healthcare activities within a health care facility using historical and contemporary tracking data to train models for predicting activity outcomes, developing pilot programs designed to favorably change the predicted outcome, and implementing the pilot programs to yield an actual outcome.

Portable electronic transmitters (e.g., RFID, RTLS, IoT, etc) are used to track locations and movements of at least some healthcare assets in the healthcare facility, thereby accumulating historic tracking data and producing contemporary (e.g., live, recent, as of last detected movement, as of last detected condition, as of last detected status change, projected, speculated, etc) tracking data. It is contemplated that at least some of the portable electronic transmitters are active transmitters, but other portable electronic transmitters can be passive transmitters, or both.

In some embodiments, locations and movements can be tracked by defining zones in the healthcare facility. The zones can also be associated with a status. For example, zone types can include a sterilization room, where asset status is dirty and not in use; storage, where asset status is clean and not in use; hallway/corridor, where asset status is not in use; operating rooms, where asset status is in use; in-patient rooms, where asset status is in use. Additionally statuses can be added by correlating the asset status and location with a patient's location and status (e.g., in-patient, out-patient, etc), as well as using patient location to determine zone status (e.g., room full status).

Most of the contemporary tracking data that is produced (and preferably at least some of the historical tracking data) is granular to a level of an individual healthcare asset within a type or category of healthcare assets. The contemporary tracking data generally tracks at least one of (a) an individual healthcare professional among a type of healthcare professional or (b) an individual healthcare equipment among a type of healthcare equipment. In some embodiments, the historical tracking data or contemporary tracking data includes at least some of the variables listed and described in Table 1.

TABLE 1

| Seq | Factor | Comments |
|---|---|---|
| 1 | OR Occupancy. This feature can be graded on a | Define OR Occupancy. (Suite |
| | 1-4 scale. 1 is less than 25%. 2 is 25%-50%. 3 is | utilization) |
| | 50% to 75% 4 is >75% | |
| 2 | Does Surgeon have prior case end time within “x” | 30 mins. (End time of the last |
| | minutes (e.g., 30) | procedure). May want to provide |
| | | 30, 60 bands |
| 3 | Does Anesthesiologist (“Anes”) have prior case end | Anes finishes case after surgeon |
| | time within “x” minutes (e.g., 45) | leaves |
| 4 | Is prior case delayed > “x” mins (e.g., 15). Use | |
| | patient in the room times. | |
| 5 | Is nurse pre-oping the patient working on more than | |
| | “x” patients (e.g., 2 patients) | |
| 6 | Does the OR suite have other rooms or have other | Notes: Case can have |
| | patients have start time within 15 mins | multiple procedures |
| 7 | Did pre op obtain all required reports , studies and | Some hospitals may have |
| | consults not less than 24 hours before the scheduled | 5PM day before as the |
| | start time | deadline |
| 8 | What time of the day is the case scheduled, (e.g., | |
| | Break day into 3 hour blocks) | |
| 9 | How many cases got scheduled on an emergency | Emergency cases typically effect |
| | basis (e.g., room number and the start times) | only one room |
| 10 | Are all lab orders processed, resulted and available | May be more relevant for ED |
| | for review. WARNING: 25% of cases labs may need | |
| | to repeated as it may not be current (e.g., dialysis | |
| | patients). Most of the time they draw and test in the | |
| | lab (e.g., ISTAT or other point of care testing). | |
| 11 | Are labs and/or test results effecting the start | |
| | time, (e.g., ISTAT is not functional) | |
| 12 | Is staffing at least “x” (e.g., 90%) of required staff | |
| 13 | How is the on-time start record for the surgeon | We are going to compare |
| | (patient into room time) due to any reason | predicted patient in to the |
| | | revised patient in time |
| 14 | Is the age of the patient more than “x’ or less than | Operating room temp needs to |
| | “y” (e.g., under 8 years old, older may not be an | be raised to the right temp for |
| | issue, elderly) | very young patients |
| 15 | Is the ASA level of patient above “x” | ASA levels from 1 to 7. Amer |
| | (e.g., 3 or higher) | soc of Anes. ASA needs to be |
| | | established as part of care plan. |
| | | 1 Healthy. 3 Sick elderly 4 |
| | | Sick. |
| | | Notes: Stable 3 vs unstable 3. |
| 16 | Are there any equipment or supply issues that are | |
| | preventing cases starting on time | |
| 17 | Does PACU have capacity | |
| 18 | Will there be delays from patients moving out of | |
| | PACU | |
| 19 | Are multiple OR rooms finishing within “x” mins | |
| | (e.g., 20) of this procedure patient in the room time; | |
| | so there may be a bottle neck for the cleaning crew | |
| 20 | Equipment failure that would lead to a delay in the | Failures are reported to charge |
| | case duration | nurse, (e.g., video camera for |
| | | laparoscopy) |
| 21 | Late patients are not tracked aggressively enough by | |
| | nurses in the holding area | |
| 22 | Surgical outcome (e.g., severe complications or | |
| | death) | |
| 23 | BMI > 30 leads to complications such as reduction in | |
| | patient's physiological ability to withstand | |
| | anesthesia and surgical stress | |
| 24 | Waiting for senior surgeon in difficult surgeries or | |
| | Medical problems required second opinion of other | |
| | specialist | |
| 25 | Patient wanted to talk to family/surgeon or in | |
| | washroom | |
| 26 | Infrastructure problems (e.g., air conditioning, | |
| | power supply, water supply, laundry) | |
| 27 | Last night emergency going on or First case is risky | |
| | surgery | |

In some embodiments, historical or contemporary data sets with healthcare asset type and location data can be used to extrapolate additional data (e.g., by models, user, etc). For example, asset usage can be extrapolated as in Table 2, below.

TABLE 2

| | | | Usage | Usage | Usage |
|---|---|---|---|---|---|
| | | | Last | Last | Last |
| Asset Type | Location | Number | Week | Month | Year |
| IV Pumps | OR | 22 | 56 | 55 | 58 |
| IV Pumps | ICU | 12 | 65 | 64 | 62 |
| IV Pumps | MAMMO | 11 | 67 | 64 | 60 |
| IV Pumps | Overall | 45 | 61 | 59.5 | 59.5 |

Likewise, asset movement patterns can also be extrapolated, as in the example in Table 3, below. In some embodiments, such information can be integrated into a heat map displaying asset location, duration, utilization, or combinations thereof.

TABLE 3

| Asset | Location | Time Enter | Time Exit | Duration | | Moved To | Status |
|---|---|---|---|---|---|---|---|
| IV Pump1 | OR | Jan 15 | Jan 15 | 60 | Mins | Storage | Not in |
| | | 10:02 AM | 11:42 AM | | | | use |
| IV Pump1 | OR | Jan 15 | Jan 15 | 3 | Mins | Hallway | Not in |
| | | 11:42 AM | 11:45 AM | | | | use |
| IV Pump1 | OR | Jan 15 | Jan 15 | 150 | Mins | OR1 | In Use |
| | | 11:45 AM | 2:15 AM | | | | |
| IV Pump1 | OR | Jan 15 | Jan 15 | 40 | Mins | Cleaning | Not in |
| | | 2:15 AM | 2:55 AM | | | Room | use |
| IV Pump1 | OR | Jan 15 | | 202 | Mins (current) | Storage | Not in |
| | | 2:55 AM | | | | | use |

| | |
|---|---|
| Distance Travelled | 300 Mtrs |
| Number of moves | 5 |
| Utilization | 20% |

It is further contemplated that information and variables related to other operations in a healthcare facility can be tracked or monitored. For example, with respect to radiology activities, variables such as patient medical record number (MRN), type of radiology procedure, the physician ordering the procedure, the nurse ordering the procedure, date and time ordered, date and time acknowledged, date and time start processing in radiology information system (RIS), date and time complete processing in RIS, or date and time communicated back to electronic medical record (EMR) are monitored. Using such variables, models can be trained to predict, for example, total number of radiology equipment, number of parallel flows that are possible, number of staff needed to operate, staff bottlenecks, total number of radiology orders placed today, total number of radiology orders currently open, weighted average for time taken for each procedure, number of equipment currently open, number of equipment currently closed, or distance of the equipment/tests from the ER.

In preferred embodiments, historical and contemporary variables monitor, and/or models predict, the factors listed in Table 4 with respect to radiology activities. It is contemplated that such factors are further used by models of the inventive subject matter to predict whether a lab result will be on time, or is delayed, with respect to real time or scheduled activities.

TABLE 4

| Seq | Factor |
|---|---|
| 1 | Load on a specific radiology service line. Is a more |
| | than 25% occupancy for a service line such as MRI. |
| | Is there 50%, 75% of 100% occupancy planned for that service line |
| 2 | Do a lot of the test currently ordered take more than the |
| | average time |
| 3 | Do a lot of patients waiting today have mobility issues. Is |
| | the acuity level for patients higher than normal |
| 4 | Is there an issue with transporters |
| 5 | Time of day. Does the lab operate at different velocities |
| | during times of the day |
| 6 | Day of week. Does the lab operate at different velocities |
| | during different days |
| 7 | Close to a break. Is the time close to a break such |
| | as a lunch break that could cause delays |
| 8 | Known closures. Are there any known closures for the lab either |
| | for repair and maintenance or some kind of a scheduled break |
| 9 | How many staff are checked in today |
| 10 | Is a particular physician on staff today that orders more |
| | than average number of tests |
| 11 | Is the load on radiology seasonal? If so are there any |
| | triggers for today |
| 12 | Are some tests more time consuming than others |
| 13 | Do tests need to be run stand alone or can they be run in a batch |
| 14 | Is there a setup/tear down process involved for (some) tests |
| 15 | Communication with transporters. Is there paging. Is there |
| | an accepted delay |

It should be appreciated that methods, models, and systems of the inventive subject matter can be used to predict outcomes of individual activities, outcomes of multiple activities (concurrent, simultaneous, staggered, or scheduled), outcomes of activities of the same type (e.g., surgery, radiology, examination, etc), outcomes of activities of different types, outcomes of activities related to a single patient, outcomes of activities related to multiple patients, or unrelated activities. For example, the outcome of a radiology activity for a patient can be individually predicted, but can also be predicted and incorporated into a multipart workflow for the patient (e.g., admittance, pre-examination, radiology, operation, post-operation, discharge, etc), a workflow of some or all scheduled radiology events in the facility, or a workflow of some or all patients in the facility.

As a further example, it is contemplated that pharmacy activities can be monitored and predicted by methods, models, and systems of the inventive subject matter. Such applications can be used to improve pharmacy services in a healthcare facility (e.g., match staffing to predicted load, etc), as well as incorporate pharmacy status into workflow modeling for other activities in the facility (e.g., operating room, emergency room, inpatient services, etc). For example, with respect to pharmacy activities, variables such as order ID, order date placed, order date filled, order date shipped, patient MRN, patient ID, provider ID, nurse ID, prescription name, prescription type, quantity, special instructions, administration type, dosage, units, refill quantity, associated risk factors, or prescription number ID can be monitored or tracked. Using such variables, models can be trained to predict, for example, total number of orders placed per day, total number of orders currently open, weighted average for time taken for each prescription, number of pharmacy personnel, number of pharmacies currently open, prescription order rate for physician, whether a prescription is seasonal, or clustering of prescriptions (e.g., strep throat is usually seasonal and will tend to have groups of patients with prescriptions clustered together, etc).

In preferred embodiments, historical and contemporary variables monitor, and/or models predict, the factors listed in Table 5 with respect to pharmacy activities.

TABLE 5

| Seq | Factor |
|---|---|
| 1 | Pharmacy Load. This feature will be graded on a 1-4 scale. 1 is |
| | less than 25%. 2 is 25%-50%. 3 is 50% to 75% 4 is >75% |
| 2 | Do a lot of the prescriptions currently ordered take more than |
| | the average time |
| 3 | How many staff are in the pharmacy currently |
| 4 | Time of day. Does the pharmacy operate at different velocities |
| | during different times of day |
| 5 | Day of week. Does the pharmacy operate at different velocities |
| | during different days |
| 6 | Close to a break. Is the time close to a break such as a lunch |
| | break that could cause delays |
| 7 | Known closures. Are there any known closures for the pharmacy |
| | either for repair and maintenance or some kind of a scheduled |
| | break |
| 8 | Is the load on pharmacy seasonal? If so are there any |
| | triggers for today |
| 9 | Do some prescriptions tend to come in batches or waves |
| 10 | Are some prescriptions more time consuming than others |

Further yet, it is contemplated that lab activities can be monitored and predicted by methods, models, and systems of the inventive subject matter. Such applications can monitor or predict the workflow and the time that a particular lab request will take, as well as incorporate duration, time, and other logistic variables into workflow modeling for other activities (preferably dependant or related activities) in the facility (e.g., operating room, emergency room, inpatient services, etc). For example, with respect to lab activities, variables such as patient MRN, type of lab ordered, group of tests, ordering physician, ordering nurse, date and time ordered, date and time acknowledged, date and time start processing in lab information systems (LIS), date and time complete processing in LIS, date and time communicated back to EMR, lab equipment used, specimen number, specimen type, or lab name can be monitored or tracked. Using such variables, models can be trained to predict, for example, total number of orders placed today, total number of orders currently open, weighted average for time taken for each procedure, number of lab personnel, number of labs currently open, number of labs currently closed, which equipment is being used, which equipment is not being used, current time to get samples into the lab, lab order rate for physician, whether some labs are seasonal, or whether some lab tests cluster (e.g., test for strep throat is usually seasonal and will tend to have groups of patients tested together, etc).

In preferred embodiments, historical and contemporary variables monitor, and/or models predict, the factors listed in Table 6 with respect to pharmacy activities.

TABLE 6

| Seq | Factor |
|---|---|
| 1 | Lab Occupancy. This feature will be graded on a 1-4 scale. |
| | 1 is less than 25%. 2 is 25%-50%. 3 is 50% to 75% 4 is >75% |
| 2 | Do a lot of the labs currently ordered take more than the |
| | average time |
| 3 | How many staff are in the lab currently |
| 4 | Time of day. Does the lab operate at different velocities |
| | during different times of day |
| 5 | Day of week. Does the lab operate at different velocities |
| | during different days |
| 6 | Close to a break. Is the time close to a break such as a |
| | lunch break that could cause delays |
| 7 | Known closures. Are there any known closures for the lab either |
| | for repair and maintenance or some kind of a scheduled break |
| 8 | is the load on labs seasonal. If so are there any triggers for today |
| 9 | Do some tests tend to come in batches or waves |
| 10 | Are some tests more time consuming than others |
| 11 | Does a test require the complete attention for the duration of |
| | the tests |
| 12 | is there a setup/teardown aspect to some tests |

In some embodiments, such variables are organized into tables for analysis (e.g., by facility personnel, by machine learning algorithms, by models, etc), as depicted in Table 7.

TABLE 7

| Lab | Lab | Average | Specimen | Data time | Date time | | | Lab | Lab |
|---|---|---|---|---|---|---|---|---|---|
| Name | Test | Time | Type | ordered | completed | Physician | Nurse | Staff | Equipment |
| ER | Strep | 20 | Saliva | 10/1/2017 | 10/1/2017 | John | Jack | Nicole | Spectrometer |
| Lab | Thoat | | | 9.00 | 9.22 | | | | |
| ER | Stomach | 15 | Blood | 10/1/2017 | 10/1/2017 | John | Jack | Manny | Infuse Pump |
| Lab | bacteria | | | 9.10 | 9.30 | | | | |

It is contemplated that models can predict, alter, or weight variables based on trends detected in historical data or as specified by a user. For example, the average time predicted for a strep throat lab is 20 minutes, but this can be altered based on additional factors related to the test, such as the time of day the test is scheduled to occur (e.g., time of day 5 am-11 am (−)2 mins, time of day 11 am-3 pm (+)5 mins, time of day 3 pm-5 pm (+)10 mins, time of day 5 pm-9 pm (−)1 min, time of day 9 pm-5 am (+)20 min) or the day of week (e.g., Mon (−)2 min, Tue-Wed (+)2 min, Thu-Fri (+)0 min, Sat-Sun (+)10 min). Further, other factors can be associated with a test (preferably by a lab test ID or name), such as the manual effort required for a test (e.g., scale of 1 as low to 10 as high), average time of the test, or the maximum number of type of lab test allowed each day (e.g., 10 strep labs, 10 stomach bacteria labs, etc). Overall lab factors can also be monitored or predicted, and incorporated into workflow models, such as staffing, lab equipment utilization, load on lab, current open orders, average manual effort required for concurrent or pending tests, etc.

The historic tracking data and contemporary tracking data are used in combination with a healthcare outcome (e.g., positive, negative, on-time, delay, fatality, recovery, remission, follow-up procedure, over budget, under budget, etc) of a previous healthcare event (e.g., workflow, treatment, operation, surgery, lab test, patient transfer, emergency procedure, etc) and at least one facility-operating model to predict a future healthcare outcome of a future healthcare event. A pilot program is then conducted by altering a planned future activity within the healthcare facility (e.g., a variable of the activity), thereby producing an actual outcome that is different from the predicted outcome. The facility-operating model is then modified to incorporate at least one alteration utilized in the pilot program and the modified facility-operating model is implemented within the healthcare facility.

In some embodiments at least one of the facility-operating models is at least partially developed by training one or more machine learning algorithms on at least part (preferably all) of the historical tracking data. The facility-operating model utilizes machine learning (e.g., at least one algorithm trained on at least one historical data set) to automatically compare at least one of (a) the actual outcome with the predicted outcome or (b) the pilot program with the actual outcome. It should be appreciated that further using at least a second facility-operating model (e.g., employs a different methodology from the first facility-operating model, is trained on a different data set, includes different limitations or degrees of freedom, etc) to comparison check a predictive accuracy of the first facility-operating model is beneficial. Such comparison can be used to adjust the first facility-operating model (e.g., adding a variable, removing a variable, adjusting the weight of a variable, etc), or another facility-operating model, or to create a new model (e.g., combining features of multiple models, removing features of a model, etc). Viewed from another perspective, it should be appreciated that the future activity (e.g., a patient variable, an activity variable, a time variable, a healthcare resource variable, etc) is altered based on a feature (e.g., a predicted outcome, a predicted risk factor, a variable identified as relevant, a variable identified as irrelevant, etc) of at least one facility-operating model.

Thus it should be appreciated that in some embodiments, the future activity is altered directly based on a second facility-operating model (e.g., a feature of the second model), or indirectly based on the second facility-operating model (e.g., a feature of the second model is combined with another model). It should also be appreciated that the future activity can be altered by part of a first model and part of a second model, either concurrently or at separated times. In some embodiments, the models are specific to activity, but it is contemplated that some models can be universally applied to any activity or workflow. Models specific to one activity can also be informationally coupled to other models specific to other activities, or networks of models can otherwise be developed to predict the outcomes of workflows. Various models can also be related hierarchically, for example where priority of models relative to each other is determined at least in part by the particular type of the model, a model for a specific activity, a specific confidence interval of the model, risk factors inherent to the model or the activity, or a combination thereof. It should be appreciated that models can be selectively trained to optimize one or more specific parameters (e.g., success rate, safety, cost, time efficiency, patient satisfaction, risk factors, etc). For example, models can determine what factors increase or decrease patient satisfaction. Once these are measured, proposed modifications can be made and implemented to drive behavior towards addressing that. As an example, if patient satisfaction is more when nurses interact with them more number of times but shorter interactions each time, nurses can be trained as such, and patient satisfaction can be reevaluated.

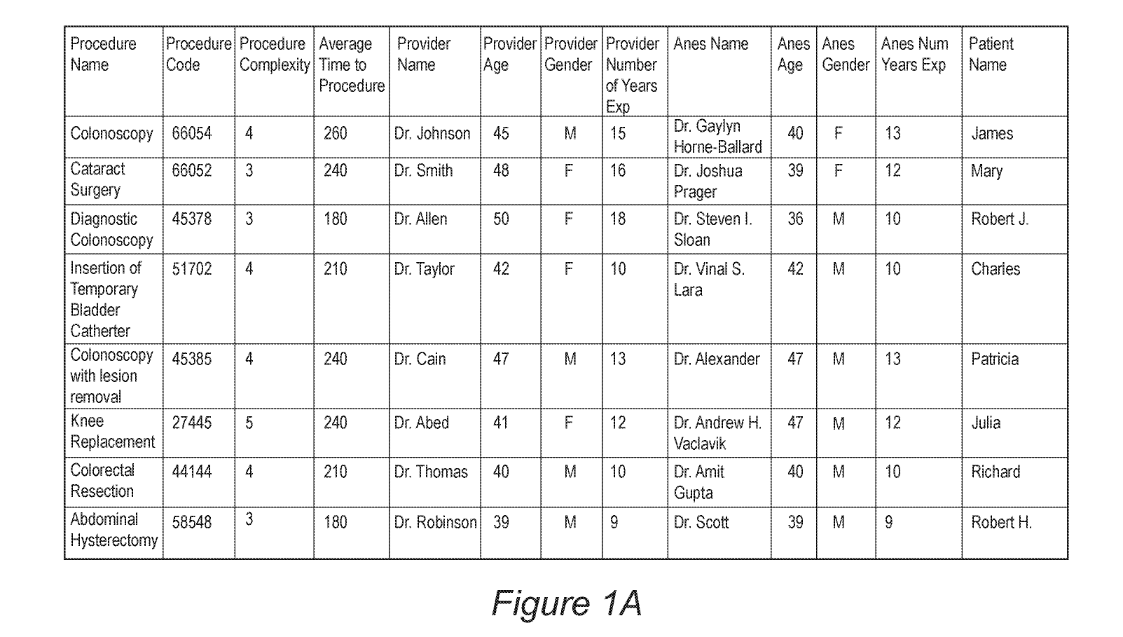

Models of the inventive subject matter (e.g., experimentation models, facility-operating models, etc) are contemplated to be trained on a variety of variables, use a variety of variables to predict outcomes, or both. Such variables include healthcare professional variables (e.g., previous case lengths, number of years of experience, age, gender, performance record, success rate, past caseload, current caseload, scheduled caseload, number of professionals, etc), patient variables (e.g., age, gender, BMI, temporary health conditions, chronic health conditions, length of stay, length of registration, length of triage, length of waiting for results, length of total nurse interaction, length of provider interaction, number of nurse interactions, number of provider interactions, time till first interaction, time till first doctor interaction, time waiting for imaging, patient updated on procedure progress, health insurance coverage, patient specific risk factors, etc), procedure variables (e.g., procedure name, procedure complexity, previous case lengths, procedure risk factors, etc), medical or lab report variables (e.g., how many reports are needed, hours before the procedure the results were completed, were the reports digitally transmitted, etc), queue variables (e.g., how many procedures ahead, time of day, day of week, real time delays, etc), equipment variables (e.g., how many equipment are needed, type of equipment, etc), machine learning variables (e.g., accuracy of model, confidence interval, successful predictions, key variables, insignificant variables, risk variables, etc), healthcare staff variables (e.g., gender, age, past caseload, current caseload, scheduled caseload, number of staff on duty, etc), and prescription variables (e.g., ordered, received, delay, availability, samples administered, samples available, etc). It should be appreciated that historical tracking data, contemporary data, or both, record some (preferably most or all) of such variables. It is contemplated that such information can be arranged in tables for analysis, as in the example at FIGS. 1A-B.

It is contemplated that at least one of (a) a status of one of the healthcare assets, (b) a status of a condition (e.g., completion of lab work, requirement for more lab work, completion of imaging, requirement for more imaging, etc) precedent to the future activity (e.g., condition is required, planned, incidental, unexpected, etc), or (c) a status of other activities in the healthcare facility is used to predict the future healthcare outcome (e.g., variable added to a facility-operating model, weight of a variable changed, training model with status, combining status with contemporary tracking data used to predict outcome, etc). In some embodiments altering the future activity entails at least one of (a) replacing a one of the healthcare assets with a second healthcare asset, (b) requisitioning an additional healthcare asset, or (c) withdrawing one of the healthcare assets from utilization in the future activity. Altering the future activity can include a number of additional or alternative changes, for example rescheduling the future activity, canceling the future activity, or prioritizing the future activity over other future activities. Viewed from another perspective, the pilot program can additionally or alternatively include rescheduling at least one other activity planned in the healthcare facility or reallocating healthcare assets related to one or more other activities.

The inventive subject matter further contemplates devices, systems, kits, and methods for orchestrating physical activities taking place in a healthcare facility. One or more historical data sets including a plurality of historic variables and a physical activity result associated with at least some of the variables are accessed. An experimentation model is trained (e.g., via machine learning, etc) on the historical data set to relate the plurality of historic variables to the physical activity result. One or more contemporary data sets related to a first plurality of contemporary variables associated with a scheduled physical activity are collected. A predicted result for the scheduled physical activity is generated by applying the contemporary data set to the experimentation model.

The predicted result is compared to a threshold (e.g., user set, default, generated by model, etc). Should the predicted result exceeds the threshold, a set of modified variables with a modification to one or more of the plurality of contemporary variables is created such that, when the set of modified variables is applied to the experimentation model, a new predicted result is generated that satisfies the threshold. The set of modified variables are then physically implemented to physically perform the scheduled activity. A new data set is recorded including (a) the implemented set of modified variables and (b) an actual result of the scheduled physical activity. The new data set, preferably in combination with the historical data set, is then used train a new experimentation model, preferably with improved accuracy of predicting results of activities. In preferred embodiments, the new experimentation model is implemented in the healthcare facility to orchestrate at least one physical activity.

In some embodiments the plurality of historical variables relates to at least two of (a) a healthcare asset data, (b) a downstream condition, (c) a patient status, (d) an upstream result, or (e) another activity that occurred in the healthcare facility. It should be appreciated that the healthcare asset data is at least one of a location, a condition, an identity, or a utilization rate of a healthcare asset. Likewise, the downstream condition is preferably at least one of a lab report, a blood test, or a radiology report. Further, the patient status is preferably at least one of late, early, prepared, a location, or a health condition status of a patient.

It is preferred the physical activity result is a patient outcome, an activity duration, an activity delay, an impact on concurrent or prospective activities in the healthcare facility, an activity cost, an activity related litigation, or a combination thereof. It should be appreciated that the experimentation model can advantageously determine a risk factor associated with each (or at least some) type of historical variables, but such risk factors can also be user defined. In such embodiments, the set of modified variables does not include modification of a variable type associated with a risk factor that exceeds a risk threshold (e.g., user defined, default, model generated, etc). For example, where the model determines modification of a variable type has a sufficient likelihood (e.g., 30%, 60%, 90%) to yield a negative patient outcome result, then that variable type will not be modified.

Likewise, it is contemplated the experimentation model (or models of the inventive subject matter) can identify a set of key variables that have a significant impact on the predicted result (e.g., determinative, 0×, 0.1×, 0.5×, 1×, 2×, 3×, 5×, 10× change yields more than 10%, 20%, 40%, 60%, 70%, 90% impact on the predicted result, likelihood of predicted result, etc). In such embodiments it is preferred the set of modified variables includes modification to at least one of the key variables identified. Similarly, it is contemplated the experimentation model can identify a set of insignificant variables that have minimal impact on the predicted result (e.g., de minimis 0×, 0.1×, 0.5×, 1×, 2×, 3×, 5×, 10× change yields less than 5%, 4%, 3%, 2%, 1% impact on the predicted result, likelihood of predicted result, etc). In such embodiments, the insignificant variables are preferably reduced or eliminated when generating the new predicted result, training the new experimentation model, implementing the new experimentation model in the healthcare facility, or a combination thereof.

In some embodiments, the models used to identify significant factors include LinearRegressionWithSGD, RidgeRegressionWithGSD, Linear Regression with Elastic Net, LASSO, or various combinations thereof.

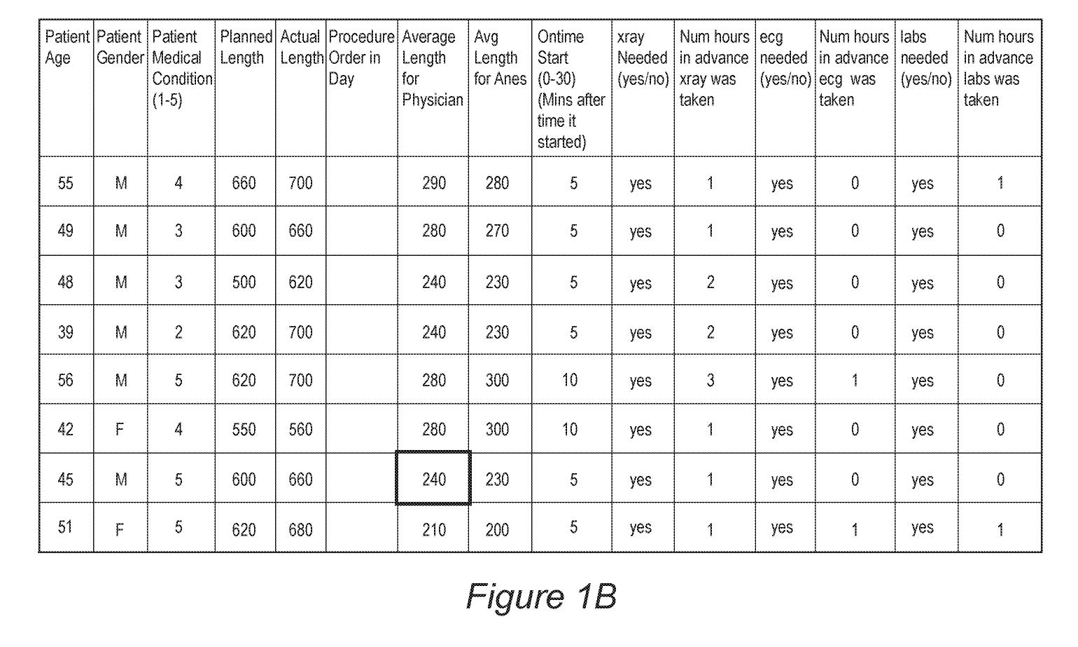

In FIG. 2, display 200 is depicted representing real time analysis of workflows in a healthcare facility. While it is anticipated that systems and methods of the inventive subject matter operate automatically, without observation or monitoring by healthcare personnel, in some applications it is advantageous to display facility workflow status as in FIG. 2. OR portion 210 of display 200 depicts Pre Op and Post Op patient occupancy (here none), as well as status of 12 available operating rooms (OR 1 through OR12). Dependency portion 220 depicts dependencies specific to a patient and the dependency status (e.g., Lab 1, Imaging 1, In Patient). Timeline 230 depicts the timeline for activities related to the operating rooms for the day and their status, with red time blocks indicated bottle necks or predicted problems. Suggestion portion 240 displays suggestions made by systems of the inventive subject matter to change the scheduled workflows or allocation of resources in the healthcare facility (e.g., Tagnos Suggestions). In preferred embodiments, the suggestions will be automatically implemented and evaluated for their impact on improving workflow.

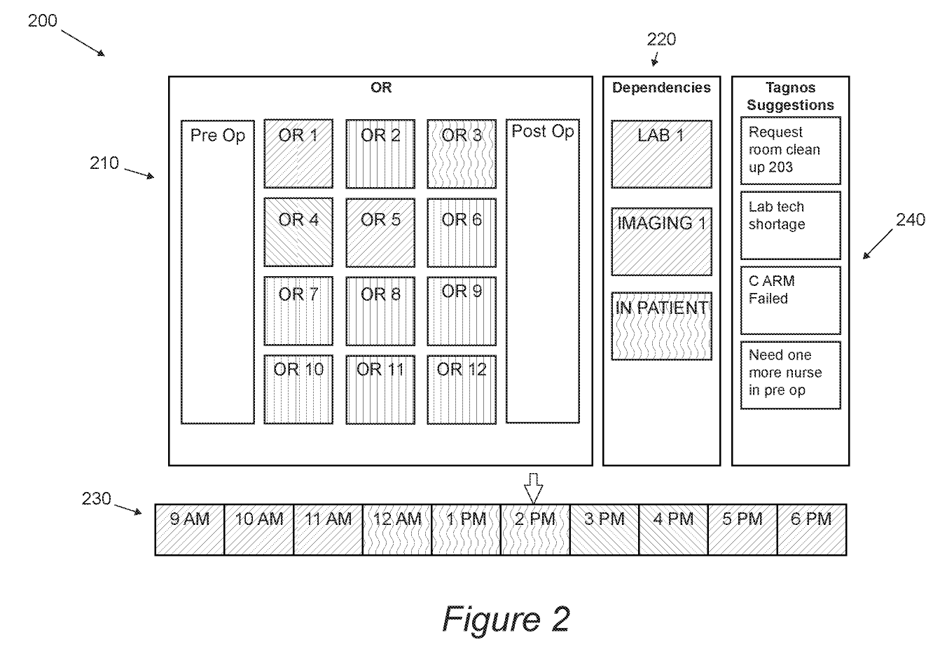

FIG. 3 depicts workflow 300 for a patient. Systems of the inventive subject matter monitor each step in the workflow in real time using variables as described above. The workflow includes registration 310, pre-op 320, surgery 330, labs 340, and recovery 350. Each step can be associated with a variety of variables and dependencies. For example, pre-op 320 is associated with predicted time (26 min), current staff (5), and bed utilization (4 of 6 utilized). Further, surgery 330 is dependent upon test results from lab 340 and operation of environmental services (EVS) 360 of the healthcare facility. Historical tracking data is analyzed to design workflows as pictured in FIG. 3, and contemporary tracking data is processed to predict likely outcomes, such as delays or bottlenecks, and propose pilot programs or modifications to be implemented.

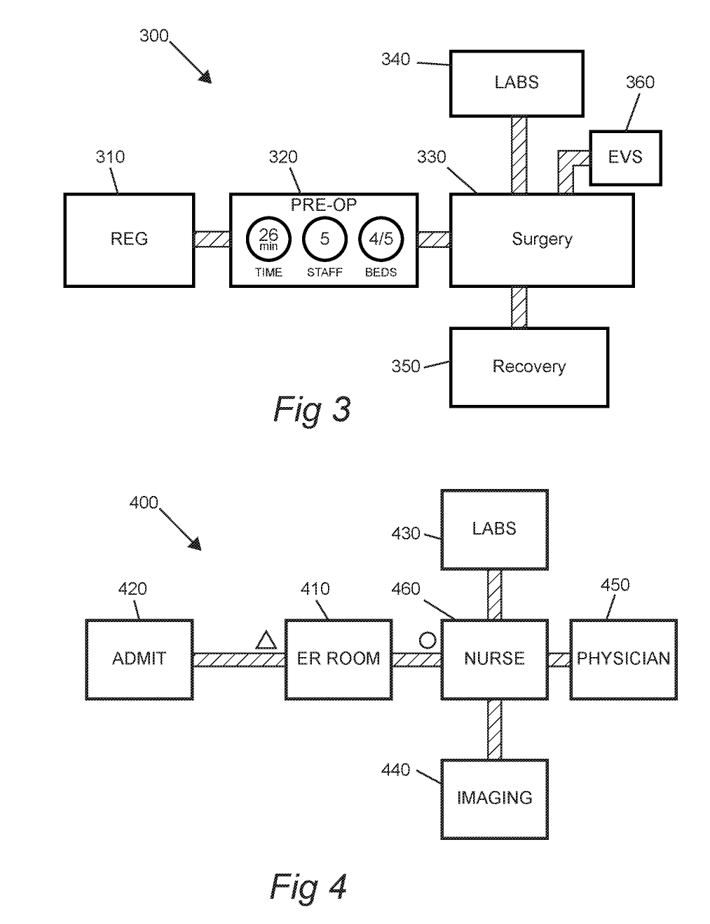

FIG. 4 depicts workflow 400 of a healthcare facility. This workflow displays the downstream and upstream dependencies for an activity in emergency room (ER) 410. In emergency room workflows, the upstream dependencies include admitting the patient (420), which can include administrative delays as well as evaluating the patient and what triage procedures to apply. The downstream dependencies include lab work 430, imaging 440, and physician 450, each of which are associated with their own variables that can speed up or delay the work flow. As most of the upstream dependencies are moderated by nurse 460 or other healthcare staff, staff variables also contribute to upstream dependencies.

It should be appreciated that ER is the gateway to at least part of many healthcare facilities, and by its nature receives patients without pre-planned appointments. This creates an environment that is ever changing. The inventive subject matter can be applied to ERs to address the chaotic nature of ER operations and derive actionable events for improving ER performance. Models, systems, and methods of the inventive subject matter use historical patterns to predict possible ER load. Historical information is analyzed to identify spikes in ER traffic and relate those spikes to detectable or predictable events or variables (e.g., time of the year, season, big holidays, sporting events, weather, etc). Viewed from another perspective, historical loads are correlated to events, and predictions are made to inform current or scheduled workflows.

In one example, models, systems, and methods of the inventive subject matter include built in interfaces to get real time information from readily available hospital feeds such as the admit, discharge, and transfer (ADT) feed. Models can be trained on the ADT data and used to predict and allocate ER patients to stages in emergency care, such as waiting room, triage, or other configurable classifications. When nurses or providers interact with the patient the information is also captured using interfaces to the current hospital systems, RFID tags, or other sensors. It is contemplated that systems can interface with nurse and provider check-in systems to keep track of who is on duty, providing real time ratio of patients to care providers. Likewise, the number of beds available and occupied can be tracked, as well as the real time through put of labs and/or radiology.

It is preferred that additional factors of healthcare activities are monitored, tracked, or used to predict outcomes, for example the following factors innate to ER activities. As ER physicians attend multiple patients and can be in variable demand, delay with one patient can cascade and should be accounted for by models. Likewise, nursing staff attend multiple patients, so if one of them falls sick it can affect the preparation for the next patient and should be accounted for. Patients usually come in with an emergency and require expedited tests. ER Rooms/bays need to be cleaned and turned over after each patient; delays in cleaning can cause downstream delays. Equipment also needs to be located and cleaned. Unexpected patient volumes can cause delays and shortages. Labs needed for tests or imaging processes may be over loaded. Beds may not be available in the recovery or in patent sections, which can cause further backups. Any of these situations can cause delays that cascade throughout the day. It should be appreciated the inventive subject matter provides many solutions and benefits, including: analyzing the current status of ER appointments, staff availability, patient movement, and predict or recognize delays in the ER system or workflows; alerting nursing staff to delays; or alerting patients and families about delays. Preferably the inventive subject matter provides suggestions on alternate routes for the staff to take to minimize the delays.

When applied to ER applications, the inventive subject matter preferably monitors, tracks, or predicts the factors in Table 8, though it should be appreciated that such factors can be advantageously considered in additional healthcare activities, in combination or in part.

TABLE 8
Factor

ER Occupancy. This feature will be graded on a 1-4 scale. 1 is less
than 25%. 2 is 25%-50%. 3 is 50% to 75% 4 is >75%
Does ER Physician more than “x” number of patients to attend to
Is nurse attending to the patient working on more than “x” patients
Are current appointments delayed in ER and there is a backup
Did multiple patients come in at the same start time
What time of the day is the appointment scheduled for (1 Early
Morning 2 Late Morning 3 Early afternoon 4 Late afternoon
Are all labs open
Is staffing at at least (“x”) 90% of required staff
How is the “quickness” rating for the Physician
Is the age of the patient more than “x’ or less than “y”
Is the health rating of the patient less than “x” factor
Is any special equipment needed that is in short supply or most
equipment that is needed is still dirty (This may depend on the procedure)
Does recovery room have capacity
Does inpatient section have capacity
Are multiple ER bays occupied and getting completed ahead of this patient
around the same time so there may be a bottle neck for the cleaning crew
What was the typical length of stay for similar complaint
What is the ETA for lab results
What is the ETA for imaging
Is appointment being transferred to another facility

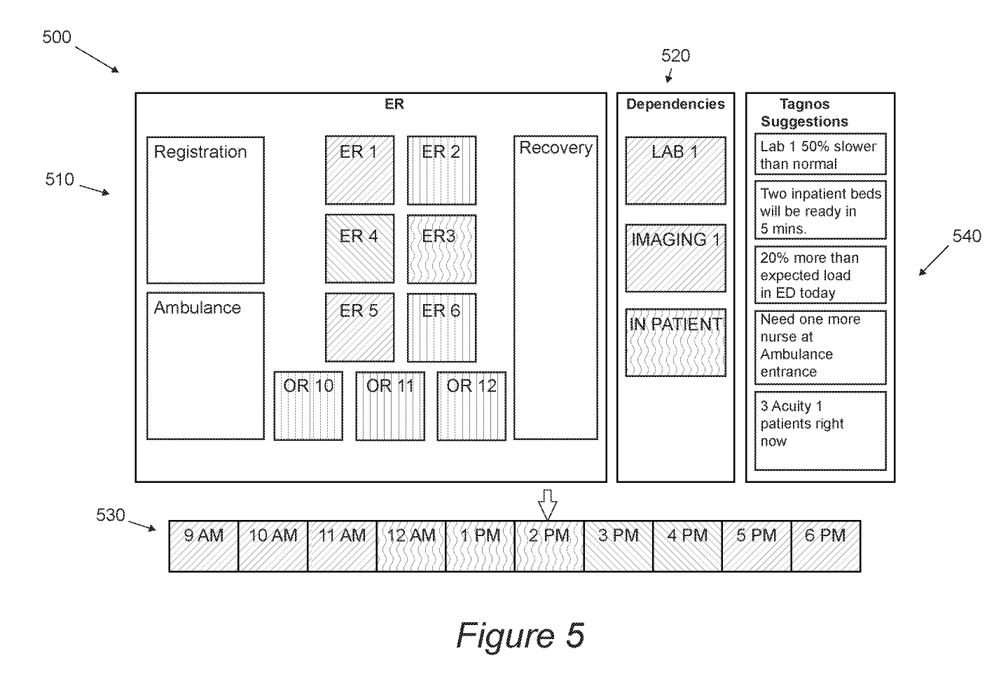

In FIG. 5, display 500 is depicted representing real time analysis of ER workflows in a healthcare facility. While it is contemplated that systems and methods of the inventive subject matter operate automatically, without observation or monitoring by healthcare personnel, in some applications it is advantageous to display facility workflow status as in FIG. 5. ER portion 510 displays ER assets, including registration area, ambulance area, recovery area, ER rooms (ER 1-6) and operating rooms (OR 10-12), with the color of each resource indicating a status level. For example, green indicates a resource that is ready, orange indicates a resource in use, red indicates a resource that is not available, and grey indicates a resource that is on standby. Dependency display 520 identifies dependency resources (Lab 1, Imaging 1, In Patient) and status, and suggestion display 540 presents suggestions from the system. Preferably, the suggestions are generated automatically by the system using models trained on historic data by analyzing contemporary data related to the ER workflows. The display also depicts timeline 530, identifying anticipated bottlenecks or issues based on analysis of contemporary data by models of the inventive subject matter.

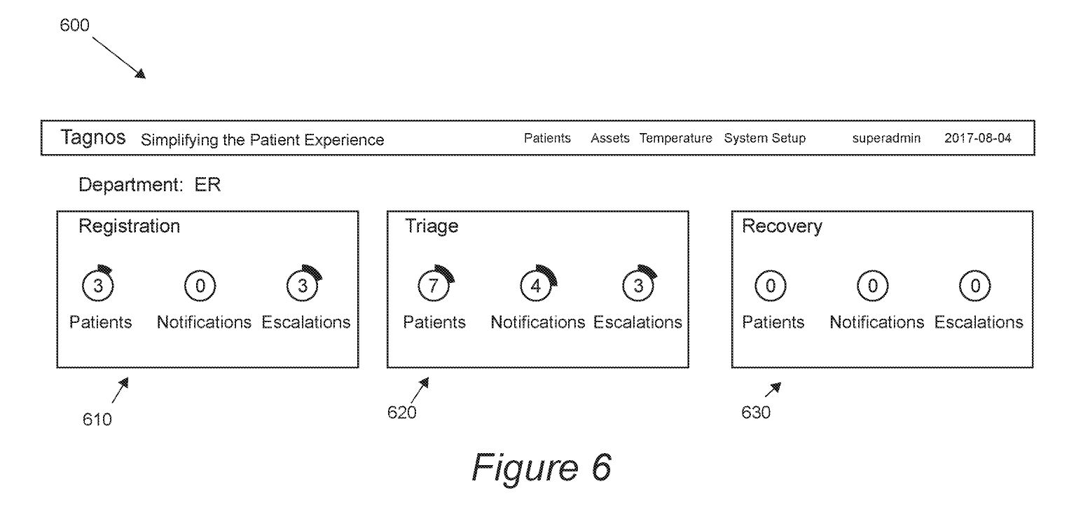

FIG. 6 depicts display interface 600. Here, registration display 610, triage display 620, and recovery display 630 of ER activities in a healthcare facility are depicted. For each stage displayed, the number of patients, number of notifications, and number of escalations are depicted, as well as the status. For example, registration display 610 depicts 3 patients with 3 escalations, indicating an upstream demand for critical ER resources. However, triage display 620 indicates 7 patients, with 4 notifications and 3 escalations, showing resources are currently saturated. As recovery display 630 indicates that no patients or resources are being used, healthcare assets such as physicians or nurses assigned to the recovery stage can be reallocated by the system to the triage stage to either activate standby triage resources or expedite treatment of patients in triage. For example, the 4 notifications in triage display 620 may indicate that 4 patients are ready to be transitioned into the recovery stage, which would free up enough triage resources to treat the patients currently in the registration stage.

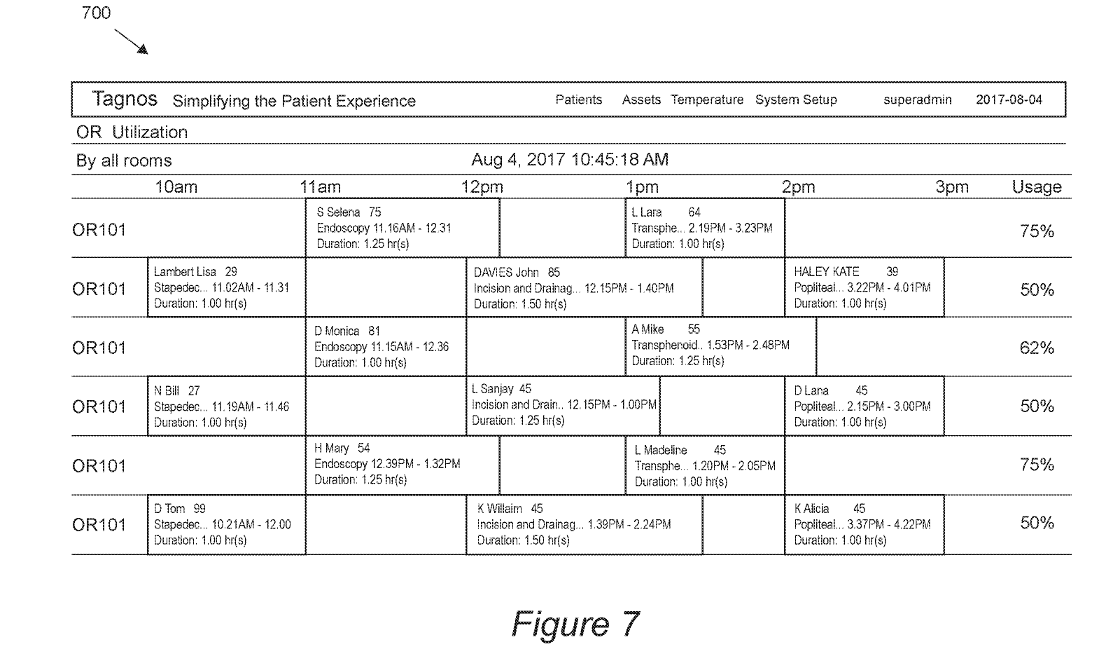

FIG. 7 depicts live schedule 700 displaying activities scheduled for a healthcare facility. The schedule is arranged along the rows by hour and the columns by operating room. Each operating room has procedures scheduled throughout the day, with each procedure identifying the patient, the type of procedure, the time scheduled for the procedure, and the time the system predicts the procedure will take based on historical and contemporary tracking data. The display also calculates the utilization rate based on the predicted and scheduled activities. The system alerts a user if a scheduled procedure is predicted to take longer than the time budgeted, for example that the stapedectomy in OR106 of D Tom is predicted to take 99 minutes, longer than the 60 minutes scheduled for the procedure. The system also informs the user of procedures that will take less time than budgeted, such as the incision and drainage in OR106 of K Willaim is predicted to take 45 minutes, less than the 90 minutes scheduled for the procedure.

Methods of orchestrating physical activities in a healthcare facility are contemplated, comprising: accessing a historical data set of a plurality of historical variables and a physical activity result; training an experimentation model to relate the plurality of historical variables to the physical activity result; collecting a contemporary data set related to a first plurality of contemporary variables for a scheduled physical activity; generating, by applying the contemporary data set to the model, a predicted result for the scheduled physical activity, wherein the predicted result exceeds a threshold; creating a set of modified variables that includes a modification to at least one of the plurality of contemporary variables, such that when the set of modified variables is applied to the experimentation model, a new predicted result satisfies the threshold; physically implementing the set of modified variables to physically perform the scheduled activity; recording a new data set comprising (a) the implemented set of modified variables and (b) an actual result of the scheduled physical activity; and training a new experimentation model using the historical data set and the new data set.

In such methods, the plurality of historical variables further relate to at least two of (a) a healthcare asset data, (b) an upstream condition, (c) a patient status, or (d) another activity that occurred in the healthcare facility. Further, (1) the healthcare asset data is at least one of a location, a condition, or a utilization rate, (2) the upstream condition is at least one of a lab report, a blood test, or a radiology report, or (3) the patient status is at least one of late, early, ready, or a health condition status.

The physical activity result is further selected from the group consisting of a patient outcome, an activity duration, an activity delay, an impact on concurrent or prospective activities in the healthcare facility, an activity cost, or an activity related litigation. The model determines a risk factor associated with each type of historical variables, and the set of modified variables does not include modification of a variable type associated with a risk factor exceeding a risk threshold. The model identifies a set of key variables having a determinative impact on the predicted result, and the set of modified variables includes modification to at least one of the key variables.

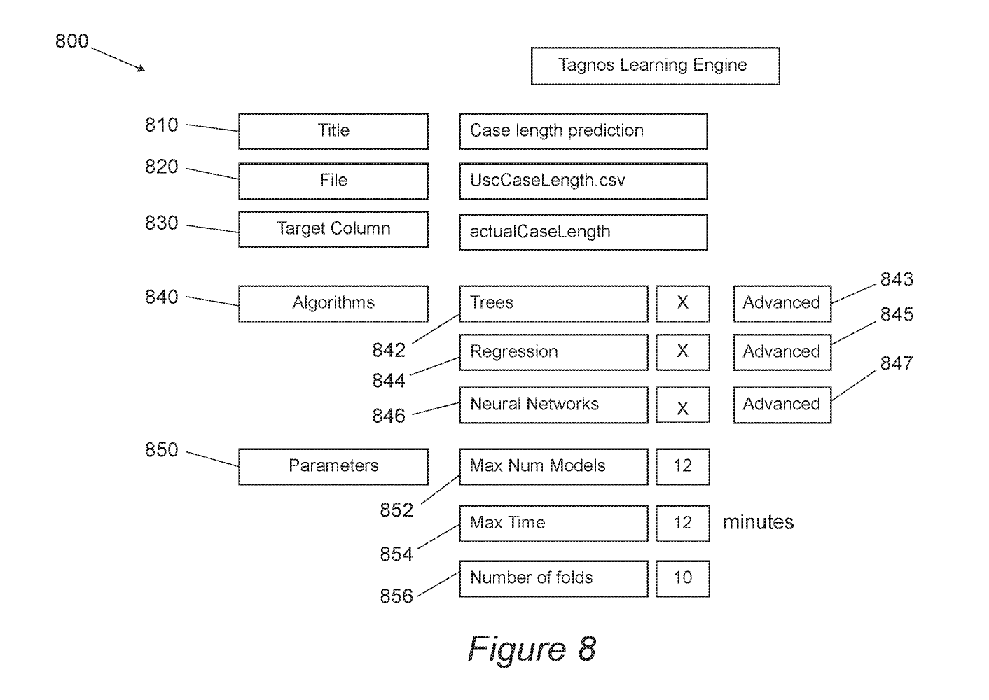

FIG. 8 depicts user interface 800 that can be used to build predictive models of the inventive subject matter. While it is desired to use a predictive model to design work flows and pilot programs to implement in enterprises and health care facilities, obtaining the best suited predictive model, or improving upon such models, is a difficult task. One aspect of the inventive subject matter is enabling enterprises and healthcare facilities to more easily and more quickly prepare accurate predictive models. User interface 800 includes provides options for a user to define criteria for selection of an accurate predictive model, including title 810, file 820, target column 830, algorithm array 840 with algorithm options 842, 844, and 846, advanced options 843, 845, and 847, and parameter array 850 with parameter options 852, 854, and 856.

Title 810 is entered by a user and in this case identifies what the objective of the model is, here to predict case length. File 820 identifies the source data (e.g., historic, contemporary, both, etc) that will be used to generate predictive models. Target column 830 identifies which column or data entry in the source data (here, UseCaseLength.csv) is the target for prediction. In this example, the actual case length variable is the value predictive models will be optimized to predict.

Algorithm array 840 includes toggles to turn on algorithm options 842 (Trees), 844 (Regression), and 846 (Neural Networks). Turning on or off the toggle selects whether the various algorithm options will be used in building predictive models. If an algorithm option is selected, the options that algorithm can be further customized using advanced options 843, 845, and 847.

Parameter array 850 can be used to select parameter options for the building and validation of models. Parameter option 852 allows a user to set the maximum number of models the system will construct and validate, for example 12. While it is possible that higher numbers of models (e.g., more than 50) permits evaluation of more permutations of models, potentially yielding more accurate models, it is contemplated that lower model counts may still yield predictive models with tolerable accuracy (e.g., within 10% accuracy 90% of the time, 90% accuracy with 5% confidence interval, etc). Parameter option 854 sets the maximum time that will be spent building and validating models, for example 12 minutes. Parameter 856 sets the number of folds that will be used to validate each model, for example 10 folds.

Increased time for modeling and validating is generally concomitant with increased numbers of models, and may yield increasingly precise or accurate models. Potential use cases that require increased precision or accuracy include planning multi-element changes to hospital protocol or institutional changes, for example redesigning workflow for multiple departments, adjusting workflows based on seasonal shifts, or adding substantial new resources to the facility (e.g., cancer ward, new wing, etc). In such cases, constructed and validated models could be more than tens of thousands, and run times could be more than a week.

However, scenarios are contemplated where models should be updated often and quickly, with less emphasis on high accuracy or precision and more emphasis on immediately improving the situation, even if it is less than a 5%, 10%, 15%, or 20% improvement. In such scenarios, it is contemplated that run times will be shorter (e.g., less than 10 min, 5 min, 2 min, 1 min, or 0.1 min) and maximum number of models will be less (e.g., less than 30 models, 20 models, 10 models, or 5 models). While such predictive models are likely less accurate and precise than models developed by analyzing thousands of models over days or weeks, they can be accurate enough to provide short term or immediate relief to difficult work flows experiencing dynamic and unpredictable changes and demands (e.g., emergency room, urgent care, widespread system failure, natural disaster, times of war, civil unrest, pandemic, lockdown, etc). Moreover, it should be appreciated that multiple or overlapping protocols can be run at the same time. For example, low model number and low run time protocols can be run successively to develop predictive models to immediately respond to problems on a short term recurring basis, while higher model number and higher run time protocols are run to develop predictive models that can develop systemic or semi-permanent solutions to the current emergency.

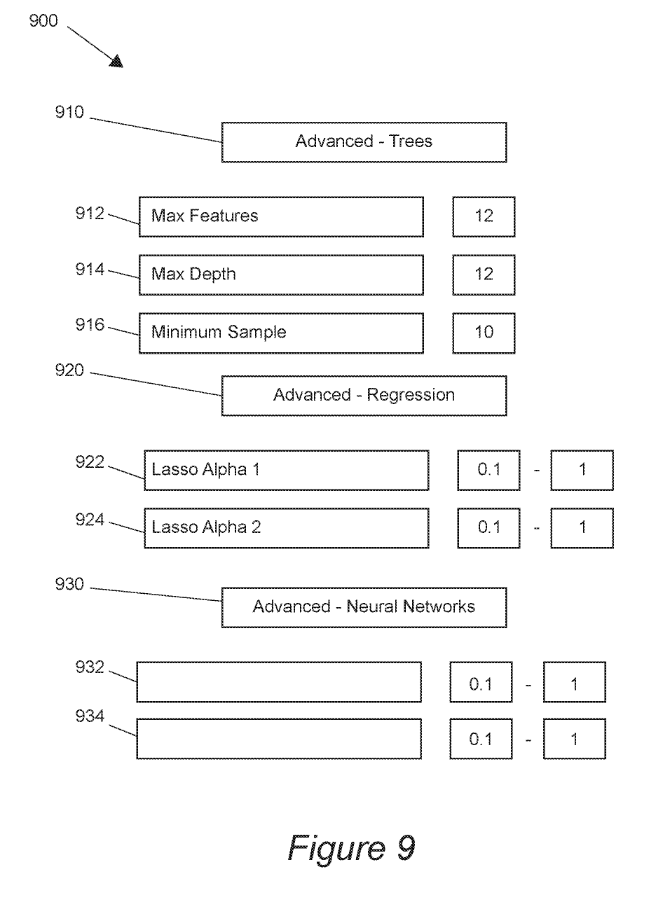

FIG. 9 depicts advanced interface 900, with advanced trees array 910 having options 912, 914, and 916, advanced regression array 920 having options 922 and 924, and advanced neural networks array 930 having options 932 and 934. With option 912 the user can set limits on the maximum number of features tree algorithms can analyze or assign, for example 12. With option 914, the user can set the maximum depth of the tree (e.g., length of longest path from a root to a leaf), for example 12. With option 916, the user can set the minimum sample size used to analyze or assign a leaf, for example 10. Using options 922 and 924, the user can set the range of lasso alpha 1 and lasso alpha 2, for example 0.1 to 1, though other options are contemplated, for example 0 to 1. Using options 932 and 934, the user can set limits on neural networks.

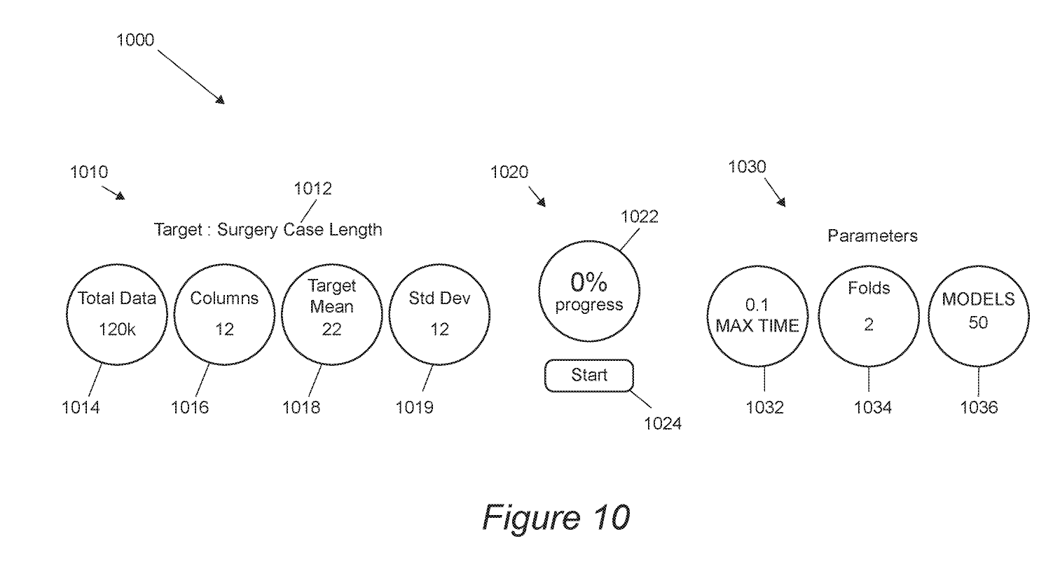

FIG. 10 depicts user interface 1000 with target array 1010, status array 1020, and parameter array 1030. Target array includes target descriptor 1012, data descriptor 1014, data descriptor 1016, target 1018, and target 1019. Target descriptor 1012 displays the user desired focus or target of the predictive models, here surgery case length. Data descriptor 1014 describes a feature of the data set that will be analyzed to generate and validate predictive models, here the size of the data set, 120,000 discrete data sets. Data descriptor 1016 further describes another feature of the data set, that the data set includes 12 columns of data. Thus, data descriptors 1014 and 1016 describe a data set comprising 12 columns and 120,000 rows, amounting to 1,440,000 data entries. Target 1018 is the objective, here that the surgery case length has a target mean of 22. Target 1019 further specifies another target the user desires, as necessary, here that the standard deviation of the surgery case length is 12.

Status array 1020 depicts the status of the system in building and validating predictive models via indicators 1022 and 1024. Here, indicator 1022 shows the progress (0%) while indicator 1024 gives the user the option to start (or stop) the system. Parameter array 1030 lists some of the parameters the system will employ to build and validate predictive models. For example, parameter 1032 specifies a maximum run time generating a model, or maximum run time for the models generated, of 0.1. While unit is not defined, it is contemplated the 0.1 hours or 0.1 minutes are appropriate units. Parameter 1034 further specifies the number of folds that will be used for cross validation, here 2, though more than 2, 4, 6, 8, 10, 20, 30, or more than 50 folds can be used for cross validation as appropriate. Parameter 1036 specifies the maximum number of models the system will build and validate, here 50, though less than 50 (e.g., 40, 30, 20, 10, 5, or less) or more than 50 (e.g. 60, 70, 80, 90, 100, 1000, 10000, 100000, or more) are contemplated.

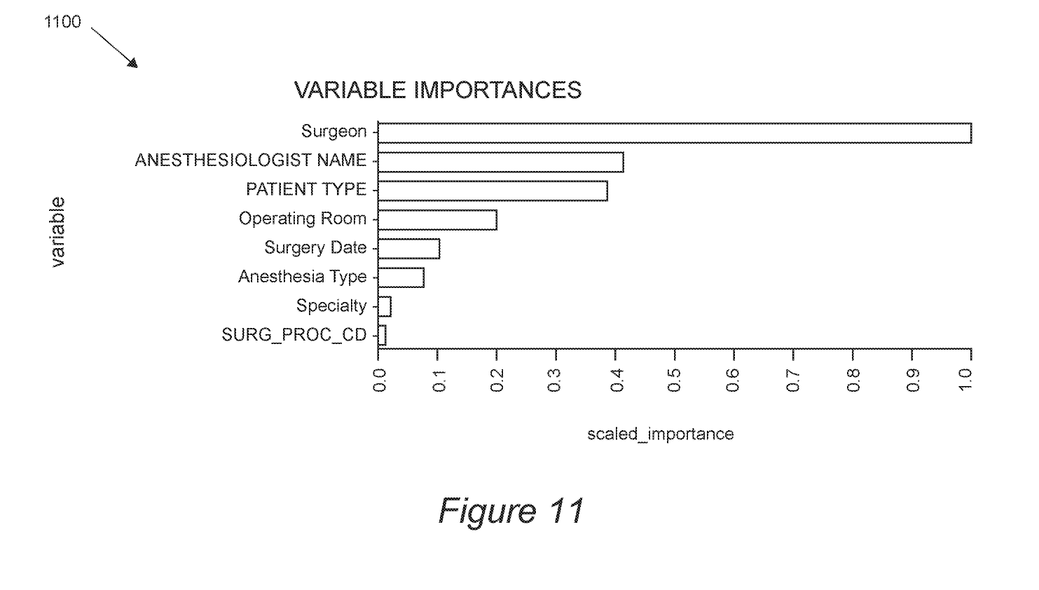

FIG. 11 depicts graph 1100 showing the scaled importance of a number of variables as evaluated and ranked by systems and methods of the inventive subject matter. The variables evaluated demonstrate some information that is considered by systems, methods, and models of the inventive subject matter, for example surgeon, anesthesiologist name, patient type, operating room, surgery date, anesthesia type, specialty, and surgical procedure code. The variables are each ranked on a scale of importance from 0.0 to 1.0, surgeon ranked as most important and surgical procedure code ranked as least important. It is contemplated that the variables are ranked by the system using a LASSO or other appropriate statistical method.

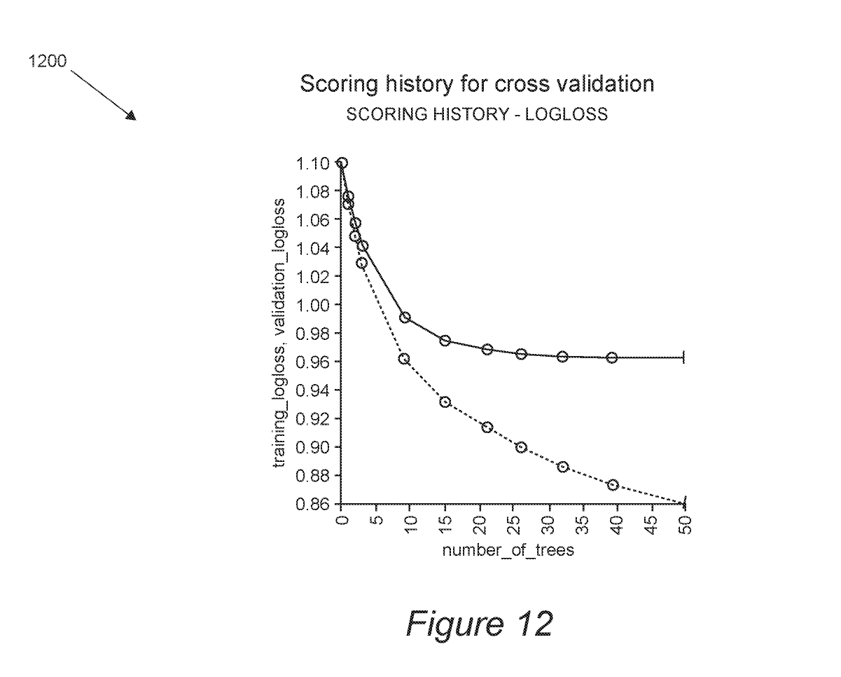

FIG. 12 depicts a graph showing the scoring history for cross validation of models built by systems and methods of the inventive subject matter. The graph indicates that for both training models and validation models, the logloss of the model is reduced as the number of trees used in the model increases, here a maximum of 50 trees.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of improving healthcare within a health care facility, comprising:
using portable electronic transmitters to track locations and movements of a plurality of healthcare assets within a healthcare facility, thereby producing historic tracking data and contemporary tracking data;
using (a) the historic tracking data, (b) the contemporary tracking data, (c) a previous healthcare outcome, and (d) a first facility-operating model to predict a future healthcare outcome;
conducting a pilot program by altering a future activity within the healthcare facility, thereby producing an actual outcome that is different from the predicted outcome;
modifying the first facility-operating model to incorporate at least one alteration utilized in the pilot program; and
implementing the modified facility-operating model within the healthcare facility.

2. The method of claim 1, wherein the contemporary tracking data is granular to a level of individual healthcare assets within a type of healthcare assets.

3. The method of claim 1, wherein the contemporary tracking data tracks at least one of (a) an individual healthcare professional among a type of healthcare professional or (b) an individual healthcare equipment among a type of healthcare equipment.

4. The method of claim 1, wherein the first facility-operating model utilizes machine learning to automatically compare at least one of (a) the actual outcome with the predicted outcome or (b) the pilot program with the actual outcome to adjust the first facility-operating model.

5. The method of claim 1, wherein the future activity is altered based on the first facility-operating model.

6. The method of claim 1, further comprising utilizing at least a second facility-operating model, which employs a different methodology from the first facility-operating model, to comparison check a predictive accuracy of the first facility-operating model.

7. The method of claim 6, wherein the future activity is altered based on the second facility-operating model.

8. The method of claim 6, wherein at least one part of the second facility-operating model is incorporated into the first facility-operating model.

9. The method of claim 1, further comprising using at least one of (a) a status of a first one of the healthcare assets, (b) a status of a condition precedent to the future activity, or (c) a status of other activities in the healthcare facility to predict the future healthcare outcome.

10. The method of claim 1, wherein the step of altering the future activity includes at least one of (a) replacing a first one of the healthcare assets with a second one of the healthcare assets, (b) requisitioning an additional healthcare asset, or (c) withdrawing a first one of the healthcare assets from utilization in the future activity.

11. The method of claim 1, wherein the contemporary tracking data comprises a movement data of a patient, and wherein the movement data of the patient is used to predict the patient is anomalous.

12. The method of claim 11, further comprising altering the future activity within the healthcare facility to mitigate the anomalous patient.

13. The method of claim 1, at least one of the historic tracking data and the contemporary tracking data comprises a data set regarding a specific healthcare professional, and wherein the data set is used to compile a profile of the specific healthcare professional.

14. The method of claim 13, wherein the data set regarding the specific healthcare professional comprises at least one of number of visits to a patient, frequency of visits with a patient, number of minutes spent with a patient, delays in visiting a patient, and amount of time spent away from a patient.

15. The method of claim 13, wherein the profile of the specific healthcare professional at least partially affects the predicted outcome, and wherein a future activity of the specific healthcare professional is modified to produce an actual outcome different from the predicted outcome.

16. A method of implementing a pilot program, comprising:
assigning an outcome for the pilot program, wherein the outcome is associated with a plurality of variables;
using at least one of a historic dataset or a contemporary dataset to (a) assign weights to the plurality of variables with respect to impact on the outcome and (b) define a set of features within the plurality of variables with respect to impact on the outcome;
constructing a plurality of predictive models for the pilot program using the weighted plurality of variables and the set of features;
cross validating the plurality of predictive models to select a validated predictive model; and
using the validated predictive model to implement the pilot program.

17. The method of claim 16, wherein the step of using the improved predictive model to implement the pilot program comprises setting a range of values for at least some of the plurality of variables that increases probability of the outcome.

18. The method of claim 16, wherein the step of using the improved predictive model to implement the pilot program comprises implementing a value for at least some of the plurality of variables to increase probability of the outcome.

19. The method of claim 16, wherein weights are assigned by a least absolute shrinkage and selection operator (LASSO) analysis.

20. The method of claim 16, wherein the set of features are used in a tree.
